(19) United States
(12) Patent Application Publication
(10) Pub. No.: US 2025/0229058 A1
(43) Pub. Date: Jul. 17, 2025

(54) CATHETER AND METHOD OF ASSEMBLING THEREOF

(71) Applicant: UNITY HEALTH TORONTO, Toronto (CA)

(72) Inventor: Christer SINDERBY, Toronto (CA)

(21) Appl. No.: 19/018,016

(22) Filed: Jan. 13, 2025

Related U.S. Application Data
(60) Provisional application No. 63/620,090, filed on Jan. 11, 2024.

(51) Int. Cl. A61M 25/00

(52) U.S. Cl. CPC A61M 25/0032; A61M 25/0009; A61M 2025/0035; A61M 2025/0037; A61M 2205/0216; A61M 2205/0233; A61M 2207/00

(57) ABSTRACT

A catheter comprising: an elongate body having: a first lumen defined in, and extending along, the elongate body, the first lumen having a first lumen cross-sectional area; a second lumen defined in, and extending along, the elongate body, the second lumen having a second cross-sectional area; and an internal wall separating the first lumen and the second lumen, the internal wall being resilient and is moveable between: a rest state; and a deflected state, in which the first lumen cross-sectional area is increased, and the second lumen cross-sectional area is decreased. In the rest state, the internal wall is curved towards the first lumen and away from the second lumen, and in the deflected state, the internal wall is curved towards the second lumen and away from the first lumen. A method of assembling the catheter.

### CROSS-REFERENCE

The present application claims priority to U.S. Provisional Application No. 63/620,090, filed on Jan. 11, 2024, entitled “CATHETER AND METHOD OF ASSEMBLING THEREOF”, the entirety of which is incorporated herein by reference.

### TECHNICAL FIELD

The present technology relates to catheters, specifically a trans-esophageal catheter which may be configured to measure diaphragm electrical activity.

### BACKGROUND

Assisted ventilation is a mode of mechanical ventilation which allows a ventilator to assist a patient with breathing. In assisted mode, the ventilator provides a breath when the patient initiates a breath such that a minimum number of breaths per minute is maintained. If the patient does not initiate a breath, the ventilator is configured to deliver breaths at a pre-set rate. Measurements of diaphragm electrical activity, via a trans-esophageal catheter configured to detect diaphragm electrical activity, provides information which may be used to improve synchronization between the patient and assisted ventilation of the ventilator.

Typically, trans-esophageal catheters used to measure diaphragm electrical activity include an array of electrodes. When detecting electrical activity, the trans-esophageal catheter is positioned such that the electrodes are aligned with the gastroesophageal junction. Signals are sent from the electrodes to an EMG system via wires (or a wire bundle) disposed within a wire lumen along a length of the catheter. Trans-esophageal catheters further include a feeding lumen to allow for feeding, providing medication, and/or emptying the stomach of the patient.

Conventional trans-esophageal catheters are designed with the wire lumen having a relatively large cross-sectional area to facilitate pull through of the wire bundle (which is often folded when pulled through). However, to accommodate the larger wire lumen, the feeding lumen will have a smaller cross-sectional area. Thus, managing the respective lumen sizes within a cross-sectional area of the catheter is important. This is especially critical with smaller trans-esophageal catheters, such as those designed for use in pre-mature and/or newborn babies (less than 1 kg), who rely on the feeding lumen of the trans-esophageal catheter to deliver food and/or medication and the detected electrical signals of the diaphragm, by the trans-esophageal catheter, to provide ventilation. Therefore, having a relatively large cross-sectional area for the wire lumen significantly limits the cross-sectional area of the feeding lumen which impacts the effectiveness of food and/or medication delivery to the pre-mature baby.

There is thus a desire to develop a catheter which overcomes the inconveniences present in the prior art.

### SUMMARY

It is an object of the present technology to ameliorate at least some of the inconveniences present in the prior art.

Broadly, there is provided a catheter having formed therein at least two lumen, such as one for accommodating wires and the other as a feeding lumen, and in which a cross-sectional area of the wire lumen can be selectively increased, such as to accommodate wire pull-through during assembly, whilst selectively decreasing a cross-sectional area of the feeding lumen. The cross-sectional areas of the first and second lumens can return to respective “at-rest” positions thereafter, therefore avoid compromising a cross-sectional area of the feeding lumen during use in the patient.

According to one aspect of the present technology, there is provided a catheter including an elongate body having: a first lumen defined in, and extending along, the elongate body, the first lumen having a first lumen cross-sectional area; a second lumen defined in, and extending along, the elongate body, the second lumen having a second cross-sectional area; and an internal wall separating the first lumen and the second lumen, the internal wall being resilient and moveable between: a rest state; and a deflected state in which the first lumen cross-sectional area is increased and the second lumen cross-sectional area is decreased.

In some embodiments, in the deflected state, the first lumen has a generally elliptical cross-section.

In some embodiments, in the deflected state, the second lumen has a generally crescent shaped cross-section with the internal wall being curved towards the second lumen.

In some embodiments, in the rest state, the second lumen has a generally semi-circular cross-section with the internal wall being curved towards the first lumen.

In some embodiments, when in the rest state, the internal wall is curved towards the first lumen and away from the second lumen.

In some embodiments, when in the deflected state, the internal wall is curved towards the second lumen and away from the first lumen. In certain embodiments, due at least in part to the configuration of the internal wall in the rest and deflected states, a stretching of the material of the internal wall is reduced or minimised compared to prior art configurations.

In some embodiments, the internal wall is substantially uniform in thickness.

In some embodiments, the internal wall moves to the deflected state in response to a force being applied onto the internal wall from an inside of the first lumen towards the second lumen.

In some embodiments, the first lumen cross-sectional area is smaller than the second lumen cross-sectional area.

In some embodiments, the catheter is composed of a polymer.

In some embodiments, the catheter is 8 Fr or less.

In some embodiments, the elongate body has a generally circular or oval-shaped cross-section.

In some embodiments, the elongate body has a major diameter of 2.24 mm when it has an oval-shaped cross-section.

In some embodiments, when the internal wall is in the deflected state, the first lumen cross-sectional area is increased up to 40% relative to the first lumen cross-sectional area in the rest state.

In some embodiments, a ratio of the first lumen cross-sectional area when the internal wall is in the rest state to the first lumen cross-sectional area when the internal wall is in the deflected state is 1:1.4.

In some embodiments, when the internal wall is in the rest state, the first lumen cross-sectional area is at least 10% of a total cross-sectional area of the elongate body.

In some embodiments, when the internal wall is in the rest state, a ratio of the first lumen cross-sectional area to a total cross-sectional area of the elongate body is 1:8.5.

In some embodiments, when the internal wall is in the deflected state, the first lumen cross-sectional area is up to 20% of a total cross-sectional area of the elongate body.

In some embodiments, when the internal wall is in the deflected state, a ratio of the first lumen cross-sectional area to a total cross-sectional area of the elongate body is 1:6.1.

In some embodiments, when the internal wall is in the deflected state, the second lumen cross-sectional area is reduced up to 20% relative to the first lumen cross-sectional area in the rest state.

In some embodiments, a ratio of the second lumen cross-sectional area when the internal wall is in the deflected state to the second lumen cross-sectional area when the internal wall is in the rest state is 1:1.1.

In some embodiments, when the internal wall is in the rest state, the second lumen cross-sectional area is at least 30% of a total cross-sectional area of the elongate body.

In some embodiments, when the internal wall is in the rest state, a ratio of the second lumen cross-sectional area to a total cross-sectional area of the elongate body is 1:2.7.

In some embodiments, when the internal wall is in the deflected state, the second lumen cross-sectional area is up to 40% of a total cross-sectional area of the elongate body.

In some embodiments, when the internal wall is in the deflected state, a ratio of the second lumen cross-sectional area to a total cross-sectional area of the elongate body is 1:3.

In some embodiments, when the internal wall is in the rest state, a ratio of the first lumen cross-sectional area to the second lumen cross-sectional area is 1:3.2.

In some embodiments, when the internal wall is in the deflected state, a ratio of the first lumen cross-sectional area to the second lumen cross-sectional area is 1:2.

In some embodiments, the catheter is an EDI catheter and further including a bundle of isolated conductors received in the first lumen during the assembly of the catheter, and wherein the application of force onto the inside of the first lumen occurs during a pull through of the bundle of isolated conductors.

In some embodiments, wherein the internal wall is configured to return to the rest state once the force is removed.

In some embodiments, the internal wall is a common wall between the first and the second lumen.

In some embodiments, the internal wall is a first internal wall; the rest state is a first internal wall rest state; and the deflected state is a first internal wall deflected state; and the elongate body further includes: a third lumen defined in, and extending along, the elongate body, the third lumen having a third lumen cross-sectional area; and a second internal wall separating the second lumen and the third lumen, the second internal wall being resilient and configured to shift between: a second internal wall rest state; and a second internal wall deflected state, in which the third lumen cross-sectional area is increased, and the second lumen cross-sectional area is decreased. In certain embodiments, in the second internal wall rest state, the second internal wall is curved towards the third lumen and away from the second lumen. In the second internal wall deflected state, the second internal wall is curved towards the second lumen and away from the third lumen.

In some embodiments, in the first and second internal wall deflected state, the first lumen and the third lumen have a generally elliptical cross-section.

In some embodiments, in the first and second internal wall deflected state, the second lumen has a generally hourglass cross-section.

In some embodiments, the first lumen cross-sectional area and the third lumen cross-sectional area are substantially similar.

In some embodiments, the second lumen cross-sectional area is larger than the first and third lumen cross-sectional area.

In some embodiments, the first internal wall moves to the first internal wall deflected state in response to a force being applied onto the first internal wall from an inside of the first lumen towards the second lumen; and the second internal wall moves to the second internal wall deflected state in response to a force being applied onto the second internal wall from an inside of the third lumen towards the second lumen.

In some embodiments, the multi-lumen catheter is composed of polyurethane.

In some embodiments the elongate body has a generally oval-shaped cross-section.

In some embodiments, the elongate body has a major diameter of 2.24 mm.

In some embodiments, at least one of: the first lumen cross-sectional area is increased by at least 60% relative to the first lumen cross-sectional area in the first internal wall rest state, when the first internal wall is in the first internal wall deflected state; and the third lumen cross-sectional area is increased by at least 60% relative to the third lumen cross-sectional area in the second internal wall rest state, when the second internal wall is in the second internal wall deflected state.

In some embodiments, at least one of: a ratio of the first lumen cross-sectional area when the first internal wall is in the first internal wall rest state to the first lumen cross-sectional area when the first internal wall is in the first internal wall deflected state is 1:1.7; and a ratio of the third lumen cross-sectional area when the second internal wall is in the second internal wall rest state to the third lumen cross-sectional area when the second internal wall is in the second internal wall deflected state is 1:1.7.

In some embodiments, at least one of: the first lumen cross-sectional area is at least 5% of a total cross-sectional area of the elongate body, when the first internal wall is in the first internal wall rest state; and the third lumen cross-sectional area is at least 5% of the total cross-sectional area of the elongate body, when the second internal wall is in the second internal wall rest state.

In some embodiments, at least one of: a ratio of the first lumen cross-sectional area to a total cross-sectional area of the elongate body is 1:17.6, when the first internal wall is in the first internal wall rest state; and a ratio of the third lumen cross-sectional area to the total cross-sectional area of the elongate body is 1:17.6, when the second internal wall is in the second internal wall rest state.

In some embodiments, at least one of: the first lumen cross-sectional area is at least 9% of a total cross-sectional area of the elongate body, when the first internal wall is in the first internal wall deflected state; and the third lumen cross-sectional area is at least 9% of the total cross-sectional area of the elongate body, when the second internal wall is in the second internal wall deflected state.

In some embodiments, at least one of: a ratio of the first lumen cross-sectional area to a total cross-sectional area of the elongate body is 1:10.4, when the first internal wall is in the first internal wall deflected state; and a ratio of the third lumen cross-sectional area to the total cross-sectional area of the elongate body is 1:10.4, when the second internal wall is in the second internal wall deflected state.

In some embodiments, at least one of: a ratio of the first lumen cross-sectional area to the second lumen cross-sectional area is 1:6, when the first internal wall is in the first internal wall rest state; and a ratio of the third lumen cross-sectional area to the to the second lumen cross-sectional area is 1:6, when the second internal wall is in the second internal wall rest state.

In some embodiments, at least one of: a ratio of the first lumen cross-sectional area to the second lumen cross-sectional area is 1:2.7, when the first internal wall is in the first internal wall deflected state; and a ratio of the third lumen cross-sectional area to the to the second lumen cross-sectional area is 1:2.7, when the second internal wall is in the second internal wall deflected state.

In some embodiments, the second lumen cross-sectional area a ratio of the second lumen cross-sectional area when the first and second internal walls are in the respective deflected states is to the second lumen cross-sectional area when the first and second internal walls are in the respective rest states is 1:1.3.

In some embodiments, the first and second internal walls are in the respective rest states, the second lumen cross-sectional area is up to 35% of a total cross-sectional area of the elongate body.

In some embodiments, when the first and second internal walls are in the respective rest states, a ratio of the second lumen cross-sectional area to a total cross-sectional area of the elongate body is 1:3.

In some embodiments, when the first and second internal walls are in the respective deflected states, the second lumen cross-sectional area is up to 26% of a total cross-sectional area of the elongate body.

In some embodiments, when the first and second internal walls are in the respective deflected states, a ratio of the second lumen cross-sectional area to a total cross-sectional area of the elongate body is 1:3.8.

In some embodiments, the catheter further includes a first bundle of isolated conductors received in the first lumen, and wherein the application of force onto the inside of the first lumen occurs during a pull through of the first bundle of isolated conductors; and a second bundle of isolated conductors received in the third lumen, and wherein the application of force onto the inside of the third lumen occurs during a pull through of the second bundle of isolated conductors.

In some embodiments, the first internal wall is configured to return to the first internal wall rest state once the force on the inside of the first lumen is removed; and the second internal wall is configured to return to the second internal wall rest state once the force on the inside of the third lumen is removed.

In another broad aspect of the present technology, a method assembling the catheter of any one of the previously described embodiments is provided. The method includes: inserting a bundle of isolated conductors into the first lumen; and pulling the bundle of isolated conductors through the first lumen, wherein in response to pulling the bundle of isolated conductors through the first lumen, the internal wall is caused to move from the rest state to the deflected state.

In some embodiments, the method further includes, subsequent to inserting the bundle of isolated conductors, folding the bundle of isolated conductors; and coupling a guiding tool to the folded bundle of isolated conductors; and wherein pulling the bundle of isolated conductors through the first lumen includes pulling on the guiding tool.

In some embodiments, the guiding tool is a guidewire and coupling the guiding tool to the folded bundle includes looping the guidewire around the folded bundle.

In some embodiments, the method further includes, in response to completion of pulling the bundle of isolated conductors through the first lumen, the internal wall is caused to move from the deflected state to the rest state.

Another broad aspect of the present technology provides a method of assembling the catheter of any one of previously described embodiments. The method includes inserting a first bundle of isolated conductors into the first lumen; pulling the first bundle of isolated conductors through the first lumen; inserting a second bundle of isolated conductors into the third lumen; and pulling the second bundle of isolated conductors into the third lumen; wherein in response to pulling the first bundle of isolated conductors through the first lumen, the first internal wall is caused to move from the first internal wall rest state to the first internal wall deflected state; and in response to pulling the second bundle of isolated conductors through the third lumen, the second internal wall is caused to move from the second internal wall rest state to the second internal wall deflected state.

Another broad aspect of the present technology provides a catheter including: an elongate body having: a first lumen defined in, and extending along, the elongate body, the first lumen having a first lumen cross-sectional area; a second lumen defined in, and extending along, the elongate body, the second lumen having a second lumen cross-sectional area; and an internal wall separating the first lumen and the second lumen, the internal wall being a common wall defining at least a portion of the first lumen and at least a portion of the second lumen, wherein: the internal wall can selectively deform to curve into the first lumen and to curve into the second lumen.

In some embodiments, when the internal wall curves into the second lumen, the second lumen cross-sectional area is decreased, and the first lumen cross-sectional area is increased.

In some embodiments, when the internal wall curves into the first lumen, the first lumen cross-sectional area is decreased, and the second lumen cross-sectional area is increased.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects, and advantages of embodiments of the present technology will become apparent from the following description, the accompanying drawings, and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 depicts a left elevation view of an elongate body of a catheter according to an embodiment of the present technology;

FIG. 2 depicts a segment of the elongate body of the catheter of FIG. 1, depicting a first lumen and second lumen defined in and extending along the elongate body, and separated by an internal wall, according to an embodiment of the present technology;

FIG. 3A depicts a cross-section view taken through line A-A of FIG. 2, depicting the internal wall in a rest state;

FIG. 3B depicts a cross-section view taken through line A-A of FIG. 2, depicting the internal wall in an intermediate state;

FIG. 3C depicts a cross-section view taken through line A-A of FIG. 2, depicting the internal wall in a deflected state;

FIG. 4A depicts a bundle of isolated conductors connected to a guiding tool;

FIG. 4B depicts the bundle of isolated conductors of FIG. 4A received in the first lumen of FIG. 2 when viewed as a cross-section view through line A-A of FIG. 2, the internal wall being in the deflected state of FIG. 3C;

FIG. 4C depicts the cross-section view of FIG. 4B after the bundle of wires has been pulled through, the internal wall returning to the rest state of FIG. 3A;

FIG. 5A depicts a cross-section view of an alternative embodiment of a catheter of FIG. 2, depicting the internal wall in a rest state, in accordance with an embodiment of the present technology;

FIG. 5B depicts a cross-section view of the catheter of FIG. 5A, depicting the internal wall in a deflected state;

FIG. 6A depicts a cross-section view of an alternative embodiment of a catheter, depicting two internal walls in a rest state, in accordance with an embodiment of the present technology;

FIG. 6B depicts a cross-section view of the catheter of FIG. 6A, depicting the two internal walls in a deflected state;

FIG. 7 depicts a flow diagram of a method of assembling the catheter of FIG. 1, in accordance with an embodiment of the present technology; and

FIG. 8 depicts a flow diagram of a method of assembling the catheter of FIGS. 6A and 6B, in accordance with an embodiment of the present technology.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, “having”, “containing”, “involving” and variations thereof herein, is meant to encompass the items listed thereafter as well as, optionally, additional items. In the following description, the same numerical references refer to similar elements.

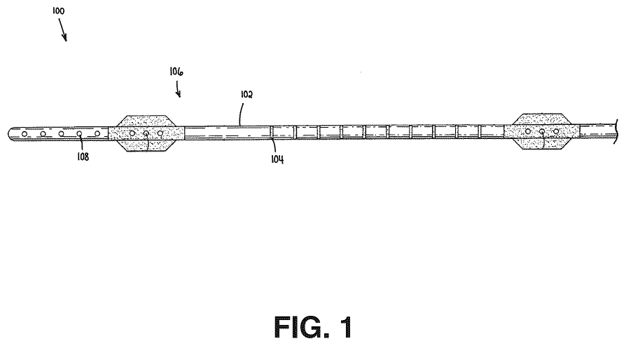

FIGS. 1 to 4C depict an embodiment of a catheter 100 of the present technology. In this embodiment, the catheter 100 is a trans-esophageal catheter 100 configured to be inserted into an esophagus of a patient for measuring diaphragm electrical activity (Edi). However, it is appreciated that, in alternative embodiments, the catheter 100 may be configured for or used in other medical procedures without departing from the scope of the present technology.

With reference to FIG. 1, the catheter 100 has an elongate body 102 having a plurality of electrodes 104 disposed along a distal portion 106. In this embodiment, the electrodes 104 are ring electrodes that are evenly distributed along a length of the distal portion 106. The catheter 100 defines a plurality of apertures 108 which are evenly distributed along a length of the distal portion 106. In some embodiments, the elongate body 102 may be composed of a biocompatible material, such as a polymer. The elongate body 102 may be flexible.

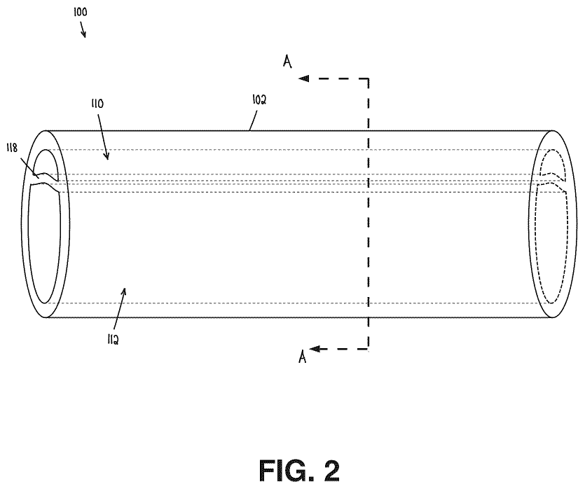

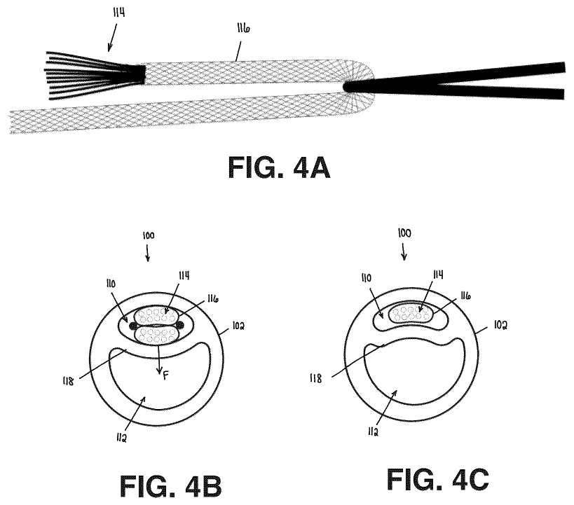

As depicted in FIGS. 2 to 4C, the elongate body 102 defines at least two lumens 110, 112. A first of the two lumens is a wire lumen 110 configured to receive a plurality of isolated conductors, such as wires 114. In this embodiment, the plurality of wires 114 are inserted as a bundle of wires 114 contained within a sheath composed of a silk material 116 (as seen in FIG. 4A), which will be described in further detail below. Each electrode 104 is connected to one end of a respective wire of the bundle of wires 114 with the other end of the wire being connectable to a monitoring system, thereby establishing electrical communication between the electrodes 104 and the monitoring system for measuring electrical activity of the diaphragm. A second of the two lumens is a feeding lumen 112 in fluid communication with the plurality of apertures 108 to enable the delivery of food and/or medication, as well as facilitate emptying a stomach of a patient. In this embodiment, the wire lumen 110 has a smaller cross-sectional area than the feeding lumen 112.

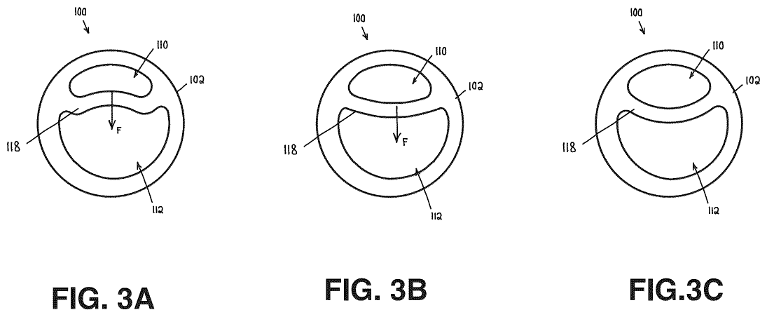

As depicted in FIG. 3A-3C, the elongate body 102 has an internal wall 118 separating the wire lumen 110 and the feeding lumen 112. In this embodiment, the internal wall 118 is a common wall defining a portion of the wire lumen 110 and a portion of the feeding lumen 112. To facilitate insertion of the bundle of wires 114 into the wire lumen 110, the internal wall 118 is configured to be resilient and moveable between a rest state (FIG. 3A) and a deflected state (FIG. 3C). In other words, the internal wall 118 is configured as a bending bridge, to selectively deform into the wire lumen 110 (FIG. 3A), such that the feeding lumen 112 has an increased cross-sectional area, and into the feeding lumen (FIG. 3C), such that the wire lumen has an increased cross-sectional area.

FIG. 3C depicts the internal wall 118 in the deflected state. When in the deflected state, the cross-sectional area of the wire lumen 110 increases to facilitate the insertion of the bundle of wires 114 into the wire lumen 110. More specifically, during insertion and pull through of the bundle of wires 114 into the wire lumen 110, the bundle of wires 114 applies a force F towards the feeding lumen 112 onto the internal wall 118 from an inside of the wire lumen 110. In response, the internal wall 118 deforms towards the feeding lumen 112, increasing the cross-sectional area of the wire lumen 110 and decreasing the cross-sectional area of the feeding lumen 112. The movement of a central portion of the internal wall 118 is radial.

FIG. 3A depicts the internal wall 118 in the rest state. Specifically, in the absence of the force F or once the force F has been removed from the internal wall 118, for example upon completion of the insertion and pull through of the bundle of wires 114 into the wire lumen 110, the resilient internal wall 118 moves back into the wire lumen 110, returning to the rest state.

The internal wall 118 being resilient and moveable between the deflected state and the rest state facilitates efficient insertion of the bundle of wires 114, during assembly of the catheter 100 without sacrificing or impeding the cross-sectional area of the feeding lumen 112 during use. That is, when in the deflected state, the size of the wire lumen 110 is temporarily increased to accommodate for pull through of the bundle of wires 114. Once the bundle of wires 114 has been pulled through, the internal wall 118 returns to the rest state, providing a larger feeding lumen to accommodate the delivery of medication and/or food, or to facilitate in emptying the stomach of the patient. This configuration of the internal wall 118 may be especially advantageous in smaller diameter catheters, such as 8 Fr or smaller, where managing the respective lumen sizes of the wire lumen 110 and the feeding lumen 112, within the cross-sectional area of the catheter 100 is important. It also enables further miniaturization of the catheter, for example, 6 Fr catheters designed for premature or newborn babies.

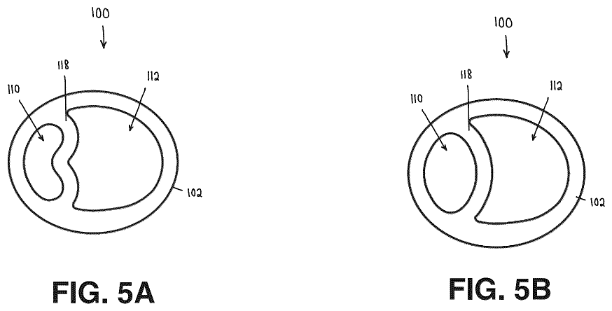

With reference to FIGS. 3A to 3C, in this embodiment, the catheter 100 has a generally circular cross-section. As previously described, the catheter 100 may be 8 Fr or less, such as 6 Fr. It is contemplated that, in alternative embodiments such as the catheter 100 depicted in FIGS. 5A and 5B, the catheter 100 has an oval-shaped cross-section, having a major diameter of 2.24 mm or less. As depicted in FIGS. 3A to 3C and 5A and 5B, the internal wall 118 is substantially uniform in thickness and curves either into the wire lumen 110 or into the feeding lumen 112. It is contemplated that, in alternative embodiments, the internal wall 118 may vary in thickness and/or may have weakened portions to promote deformation of the internal wall 118.

With specific reference to FIGS. 3A and 5A, the rest state of the internal wall 118 will be described in further detail. When in the rest state, the internal wall 118 curves into the wire lumen 110 and away from the feeding lumen 112, such that the feeding lumen 112 has a generally semi-circular cross-section. In other words, the internal wall 118 is concave, curving inwardly. In some embodiments, the cross-sectional area of the wire lumen 110 is at least 10% of a total cross-sectional area of the elongate body 102. A ratio between the cross-sectional area of the wire lumen 110 and the total cross-sectional area of the elongate body 102 is 1:8.5. In some embodiments, the cross-sectional area of the feeding lumen 112 is at least 30% of the total cross-sectional area of the elongate body 102. A ratio between the cross-sectional area of the feeding lumen 112 and the total cross-sectional area of the elongate body 102 is about 1:2.7. In some embodiments, a ratio between the cross-sectional area of the wire lumen 110 and the cross-sectional area of the feeding lumen 112 is about 1:3.2.

With specific reference to FIGS. 3C and 5B, the deflected state of the internal wall 118 will be described in further detail. When in the deflected state, the internal wall 118 curves into the feeding lumen 112 and away from the wire lumen 110, increasing the cross-sectional area of the wire lumen 110. In other words, the internal wall 118 is convex, curving outwardly. In these embodiments, the wire lumen 110 has a generally elliptical cross-section and the feeding lumen 112 has a generally crescent-shaped cross-section. In some embodiments, the cross-sectional area of the wire lumen 110 is increased up to 40%. A ratio of the cross-sectional area of the wire lumen 110 in the deflected state, compared to the rest state, is about 1:1.4. The cross-sectional area of the feeding lumen 112 is decreased up to 20%. A ratio of the cross-sectional area of the feeding lumen 112 in the deflected state, compared to the rest state, is about 1:1.1. In some embodiments, the cross-sectional area of the wire lumen 110 is up to 20% of the total cross-sectional area of the elongate body 102. A ratio of the cross-sectional area of the wire lumen 110 to the total cross-sectional area of the elongate body 102 is about 1:6.1. In some embodiments, the cross-sectional area of the feeding lumen 112 is up to 40% of the total cross-sectional area of the elongate body 112. A ratio of the cross-sectional area of the feeding lumen 112 to the total cross-sectional area of the elongate body 102 is about 1:3. In some embodiments, a ratio of the cross-sectional area of the wire lumen 110 and the cross-sectional area of the feeding lumen 112 is about 1.2.

It is appreciated that, although the rest state and the deflected state of the internal wall 118 has been described, the internal wall 118 may travel through intermediate states, one of which is depicted in FIG. 3B.

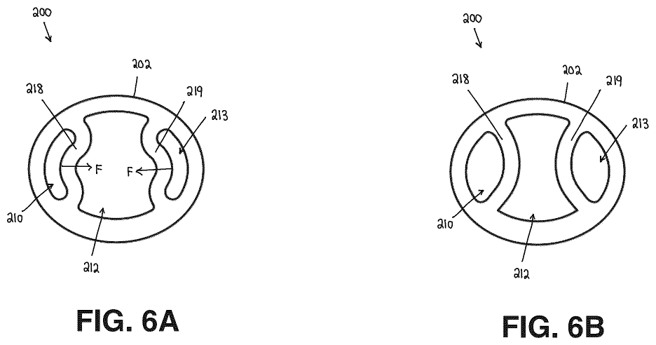

With reference to FIGS. 6A and 6B, an alternative embodiment of a catheter 200 is depicted. In this embodiment, the catheter 200 has an elongate body 202 which is configured similarly to the elongate body 102 of the catheter 100, and therefore will not be described in further detail. The elongate body 202 defines three lumens 210, 212, 213 extending along the elongate body 202. In this embodiment, the first of the three lumens 210 and the third of the three lumens 213 each define a wire lumen 210, 213 and the second of the three lumens 212 defines a feeding lumen 212. In this embodiment, the first wire lumen 210 and the second wire lumen 213 are substantially similar in size. In other words, the wire lumens 210, 213 have similar cross-sectional areas. Each of the cross-sectional areas of the wire lumens 210, 213 are smaller than a cross-sectional area of the feeding lumen 212. The elongate body 202 has a first internal wall 218 separating the first wire lumen 210 from the feeding lumen 212. The elongate body 202 has a second internal wall 219 separating the second wire lumen 213 from the feeding lumen 212.

As previously described, the internal walls 218, 219 are resilient and moveable between a rest state and a deflected state. That is, the internal walls 218, 219 are each configured as a bending bridge to selectively deform into the respective wire lumen 210, 213 (FIG. 6A), such that the feeding lumen 212 has an increased cross-sectional area, and into the feeding lumen 212 (FIG. 6B), such that each of the wire lumens 210, 213 have an increased cross-sectional area.

FIG. 6B depicts the internal walls 218, 219 in the deflected state. When in the deflected state, the cross-sectional areas of each of the wire lumens 210, 213 increases to facilitate insertion/pull-through of a bundle of wires 214, 215 into each wire lumen 210, 213, respectively. During insertion and pull through of the first bundle of wires 214 into the first wire lumen 210, a force F is applied to the first internal wall 218 towards the feeding lumen 212 from an inside of the first wire lumen 210. In response, the first internal wall 218 deforms towards the feeding lumen 212, increasing the cross-sectional area of the first wire lumen 210. Similarly, during insertion and pull through of the second bundle of wires 214 into the second wire lumen 213, a force F is applied to the second internal wall 219 towards the feeding lumen 212 from an inside of the second wire lumen 213. In response, the second internal wall 219 deforms towards the feeding lumen 212, increasing the cross-sectional area of the second wire lumen 213.

FIG. 6A depicts the internal walls 218, 219 in the rest state. Specifically, in the absence of force F or once the force F has been removed from each of the internal walls 218, 219, for example upon completion of the insertion and pull through of each of the bundle of wires 214, 215, the resilient internal walls 218, 219 deform back into the respective wire lumens 210, 213, returning to the rest state.

With continued reference to FIGS. 6A and 6B, the catheter 200 has a generally oval cross-section with a major diameter of 2.24 mm or less. In this embodiment, the internal walls 218, 219 are substantially uniform in thickness and curves either into a respective wire lumen 210, 213 or into the feeding lumen 212. In this embodiment, the first internal wall 218 and the second internal wall 219 are substantially similar to one another. However, it is contemplated that, in alternative embodiments, the internal walls 218, 219 may be configured different from one another. It is further contemplated that, in other embodiments, the internal walls 218, 219 may vary in thickness and/or may have weakened portions to promote deformation of the internal walls 218, 219.

With reference to FIG. 6A, the rest state of the internal walls 218, 219 will now be described in further detail. When in the rest state, the first internal wall 218 curves into the first wire lumen 210 and the second internal wall 219 curves into the second wire lumen 213. In other words, the first and second internal walls 218, 219 are concave, curving inwardly. Each of the internal walls 218, 219 curve away from the feeding lumen 212. In some embodiments, the cross-sectional area of the first wire lumen 210 is at least 5% of a total cross-sectional area of the elongate body 202. A ratio of the cross-sectional area of the first wire lumen 210 to the total cross-sectional area of the elongate body 202 is about 1:17.6. In some embodiments, a ratio of the cross-sectional area of the first wire lumen 210 to the cross-sectional area of the feeding lumen 212 is 1:6. In some embodiments, the cross-sectional area of the feeding lumen 212 is up to 35% of the total cross-sectional area of the elongate body 202. A ratio between the feeding lumen 212 and the total cross-sectional area of the elongate body 202 is about 1:3. As previously described, the first and second wire lumens 210, 213 are substantially similar in cross-sectional area and, thus, the percentages and ratios described with respect to the first wire lumen 210 are applicable to the second wire lumen 213 and will not be described in further detail.

With reference to FIG. 6B, the deflected state of the internal walls 218, 219 is depicted and will now be described in further detail. When in the deflected state, each of the internal walls 218, 219 curve into the feeding lumen 212 and away from the respective wire lumens 210, 213, increasing the cross-sectional area of the wire lumens 210, 213 and decreasing the cross-sectional area of the feeding lumen 212. In other words, the first and second internal walls 218, 219 are convex, curving outwardly. In this embodiment, the wire lumens 218, 219 have a general elliptical cross-section and the feeding lumen 212 has a generally hourglass cross-section. In some embodiments, the cross-sectional area of the first wire lumen 210 is increased by at least 60% relative to the cross-sectional area of the first wire lumen 210 in the rest state. A ratio of the cross-sectional area of the first wire lumen 210 to the cross-sectional area of the first wire lumen 210 in the rest state is about 1:1.7. In some embodiments, the cross-sectional area of the first wire lumen 210 is at least 9% of the total cross-sectional area of the elongate body 202. A ratio of the cross-sectional area of the first wire lumen 210 to the total cross-sectional area of the elongate body 202 is about 1:10.4. In some embodiments, a ratio of the cross-sectional area of the first wire lumen 210 and the cross-sectional area of feeding lumen 212 is about 1:2.7. In some embodiments, a ratio of the cross-sectional area of the feeding lumen 212 when in the deflected state to the cross-sectional area of the feeding lumen 212 when in the rest state is 1:1.3. In some embodiments, the feeding lumen 212 is decreased by at least 22%. In some embodiments, the cross-sectional area of the feeding lumen 212 is up to 26% of the total cross-sectional area of the elongate body 202. A ratio of the cross-sectional area of the feeding lumen 212 to the total cross-sectional area of the elongate body 202 is about 1:38. As previously described, the first and second wire lumens 210, 213 are substantially similar in cross-sectional area and, thus, the percentages and ratios described with respect to the first wire lumen 210 are applicable to the second wire lumen 213 and will not be described in further detail.

It is appreciated that, in some instances, only a single internal wall 218, 219 of the catheter 200 may deflect. For example, if only one bundle of wires 214 was pulled through the first wire lumen 210, while the other wire lumen 213 remained empty.

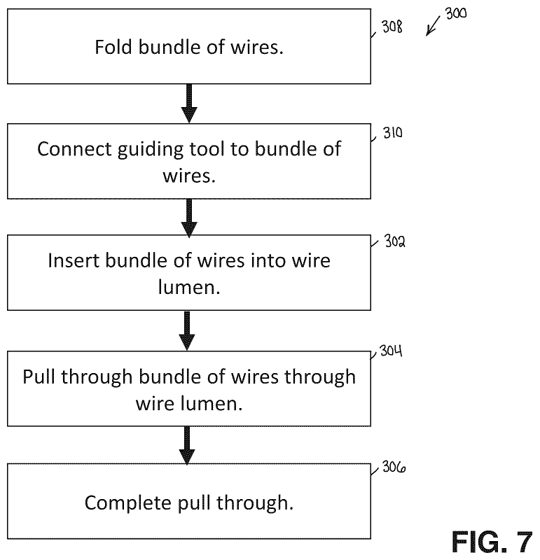

With reference to FIGS. 4A to 4C and FIG. 7, a method of assembling the catheter 100 will now be described in detail.

The method 300 begins, at step 302, with inserting the bundle of wires 114 into the wire lumen 110. At step 304, the bundle of wires 114 are pulled through the wire lumen 110 which causes the internal wall 118 to move from the rest state to the deflected state. The method continues, at step 306, of completing pull through of the bundle of wires 114. In response to completing the pull through, the internal wall 118 moves from the deflected state to the rest state.

In some embodiments, the individual wires of the bundle of wires 114 are configured to redistribute themselves, such as by spreading, within the sheath 116. In other words, once the pull through of the bundle of wires 114 has been completed, a cross-sectional shape of the bundle of wires 114 widens and flattens as the internal wall 118 deforms into the wire lumen 110. In other embodiments, a position of the wires within the bundle of wires 114 is fixed and the individual wires do not re-distribute.

In some embodiments, prior to step 302 of inserting the bundle of wires 114 into the wire lumen 110, the method 300 optionally includes step 308 of folding the bundle of wires 114 and step 310 of coupling a guiding tool 120 to the folded bundle of wires 114 (as depicted in FIG. 4A). In this embodiment, the guiding tool 120 is a guidewire which is looped around the folded bundle of wires 114. The folded bundle of wires 114 is then pulled through the wire lumen 110 (as depicted in FIG. 4B). Upon completing the pull through, the bundle of wires 114 are unfolded and, thus, the bundle of wires 114 remains within the wire lumen 110 in an unfolded state (as depicted in FIG. 4C).

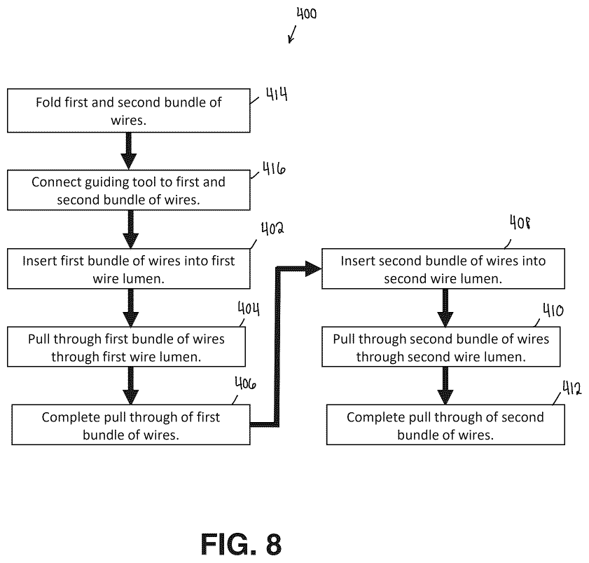

With reference to FIG. 8, a method 400 of assembling the catheter 200 will now be described in detail. By assembling is meant positioning the bundle of wires 214 in the wire lumen 110 in an elongated, unfolded state. This embodiment includes step 402 of inserting the bundle of wires 214 into the wire lumen 210, step 404 of pulling the bundle of wires 214 through the wire lumen 210 which, in response, causes the first internal wall 218 to deform into the deflected state, and step 406 of completing the pull through of the bundle of wires 214 which, in response, causes the first internal wall 218 to return to the rest state. The method 400 further includes step 408 to 412 regarding inserting the second bundle of wires 215 into the second wire lumen 213 which are similar to steps 402 to 406 and, therefore, will not be described in further detail.

It is appreciated that, in some embodiments, steps 402 to 406 may be performed in parallel with steps 408 to 412, or consecutively as described.

Similar to previously described method 300, prior to step 402 and 408 of inserting the bundle of wires 214, 215 into the respective wire lumens 210, 213, the method 400 optionally includes step 414 of folding the bundle of wires 214, 215 and step 416 of coupling a guiding tool 120 to each of the folded bundle of wires 214, 215. In this embodiment, the guiding tool 120 is a guidewire which is looped around each of the folded bundle of wires 214, 215. The folded bundle of wires 214, 215 are then pulled through the respective wire lumens 210, 213. Upon completing the pull through, the bundle of wires 214, 215 are unfolded and, thus, the bundle of wires 214, 215 remains within the respective wire lumens 210, 213 in an unfolded state.

It is appreciated that, although the disclosed embodiments described catheters 100, 200 for detecting Edi, the catheters 100, 200 may be configured for other uses without departing from the scope of the present technology.

As presented herein, the disclosed embodiments provide catheters 100, 200. The embodiments presented herein disclose catheters 100, 200 having internal walls 118, 218, 219 moveable between the rest state and the deflected state. Specifically, when in the rest state, the internal walls 118, 218, 219 are configured to curve into a respective wire lumen 110, 210, 213 and away from the feeding lumen 112, 212. When in the deflected state, the internal walls 118, 218, 219 are configured to curve into the feeding lumen 112, 212 and away from the respective wire lumen 110, 210, 213. As a result, when in the deflected state, the wire lumens 110, 210, 213 have an increased cross-sectional area to facilitate insertion and pull through of the bundle of wires 114, 214, 215 through the wire lumens 110, 210, 213. This provides various benefits, especially when the catheters 100, 200 have smaller cross-sectional areas (e.g., as those intended for premature and/or newborn babies). These benefits include, but are not limited to, temporarily increasing the size of the wire lumens 110, 210, 213 to facilitate the insertion and pull through of the bundle of wires 114, 214, 215 and, once pull through has been completed, decreasing the size of the wire lumens 110, 210, 213 to provide a larger feeding lumen 112, 212 which is used to deliver medicine and/or food and/or to empty the stomach of the patient during use.

Modifications and improvements to the above-described embodiments of the present invention may become apparent to those skilled in the art. The foregoing description is intended to be exemplary rather than limiting. The scope of the present invention is therefore intended to be limited solely by the appended claims.

What is claimed is:

1. A catheter comprising:
an elongate body having:
a first lumen defined in, and extending along, the elongate body, the first lumen having a first lumen cross-sectional area;
a second lumen defined in, and extending along, the elongate body, the second lumen having a second cross-sectional area; and
an internal wall separating the first lumen and the second lumen, the internal wall being resilient and is moveable between:
a rest state; and
a deflected state, in which the first lumen cross-sectional area is increased, and the second lumen cross-sectional area is decreased, wherein:
in the rest state, the internal wall is curved towards the first lumen and away from the second lumen, and
in the deflected state, the internal wall is curved towards the second lumen and away from the first lumen.

2. The catheter of claim 1, wherein, in the deflected state, the first lumen has a generally elliptical cross-section.

3. The catheter of claim 2, wherein, in the deflected state, the second lumen has a generally crescent shaped cross-section with the internal wall being curved towards the second lumen.

4. The catheter of claim 1, wherein, in the rest state, the second lumen has a generally semi-circular cross-section with the internal wall being curved towards the first lumen.

5. The catheter of claim 1, wherein the internal wall is substantially uniform in thickness.

6. The catheter of claim 1, wherein the internal wall moves to the deflected state in response to a force being applied onto the internal wall from an inside of the first lumen towards the second lumen.

7. The catheter of claim 1, wherein the lumen catheter is 8 Fr or less.

8. The catheter of claim 1, wherein the elongate body has a generally circular cross-section.

9. The catheter of claim 1, wherein the elongate body has a generally oval-shaped cross-section and a major diameter of 2.24 mm.

10. The catheter of claim 1, wherein, when the internal wall is in the deflected state, the first lumen cross-sectional area is increased up to 40% relative to the first lumen cross-sectional area in the rest state.

11. The catheter of claim 1, wherein, when the internal wall is in the rest state, the first lumen cross-sectional area is at least 10% of a total cross-sectional area of the elongate body.

12. The catheter of claim 1, wherein, when the internal wall is in the deflected state, the first lumen cross-sectional area is up to 20% of a total cross-sectional area of the elongate body.

13. The catheter of claim 1, wherein when the internal wall is in the rest state, the second lumen cross-sectional area is at least 30% of a total cross-sectional area of the elongate body.

14. The catheter of claim 1, wherein the catheter is an EDI catheter and further comprising a bundle of isolated conductors received in the first lumen during the assembly of the catheter, and wherein the application of force onto the inside of the first lumen occurs during a pull through of the bundle of isolated conductors.

15. The catheter of claim 1, wherein the internal wall is configured to return to the rest state once the force is removed.

16. The catheter of claim 1, wherein the internal wall is a common wall between the first and the second lumen.

17. The catheter of any one of claim 1, wherein:
the internal wall is a first internal wall;
the rest state is a first internal wall rest state; and
the deflected state is a first internal wall deflected state; and
the elongate body further comprises:
a third lumen defined in, and extending along, the elongate body, the third lumen having a third lumen cross-sectional area; and
a second internal wall separating the second lumen and the third lumen, the second internal wall being resilient and configured to shift between:
a second internal wall rest state; and
a second internal wall deflected state, in which the third lumen cross-sectional area is increased, and the second lumen cross-sectional area is decreased, wherein:
in the second internal wall rest state, the second internal wall is curved towards the third lumen and away from the second lumen; and
in the second internal wall deflected state, the second internal wall is curved towards the second lumen and away from the third lumen.

18. A method of assembling a catheter comprising:
an elongate body having:
a first lumen defined in, and extending along, the elongate body, the first lumen having a first lumen cross-sectional area;
a second lumen defined in, and extending along, the elongate body, the second lumen having a second cross-sectional area; and
an internal wall separating the first lumen and the second lumen, the internal wall being resilient and is moveable between:
a rest state; and
a deflected state, in which the first lumen cross-sectional area is increased, and the second lumen cross-sectional area is decreased, wherein:
in the rest state, the internal wall is curved towards the first lumen and away from the second lumen, and
in the deflected state, the internal wall is curved towards the second lumen and away from the first lumen, the method comprising:
inserting a bundle of isolated conductors into the first lumen; and
pulling the bundle of isolated conductors through the first lumen,
wherein in response to pulling the bundle of isolated conductors through the first lumen,
the internal wall is caused to move from the rest state to the deflected state.

19. The method of claim 18, further comprising:
subsequent to inserting the bundle of isolated conductors, folding the bundle of isolated conductors; and
coupling a guiding tool to the folded bundle of isolated conductors; and
wherein pulling the bundle of isolated conductors through the first lumen comprises pulling on the guiding tool.

20. A catheter comprising:
an elongate body having:
a first lumen defined in, and extending along, the elongate body, the first lumen having a first lumen cross-sectional area;
a second lumen defined in, and extending along, the elongate body, the second lumen having a second lumen cross-sectional area; and
an internal wall separating the first lumen and the second lumen, the internal wall being a common wall defining at least a portion of the first lumen and at least a portion of the second lumen, wherein:
the internal wall can selectively deform between a first configuration in which it curves into the first lumen and away from the second lumen, and a second configuration in which it to curves into the second lumen and away from the first lumen.
